(19) United States
(12) Patent Application Publication
(10) Pub. No.: US 2013/0321607 A1
(43) Pub. Date: Dec. 5, 2013

(54) MONITORING SYSTEM FOR CONTROLLING THE PAN/TILT OF A CAMERA USED IN A SURGERY ROOM

(71) Applicants: Pierre de Boucherville, Montreal (CA); Goran Matic, Beaconsfield (CA); Pierre Lepage, Drummondville (CA)

(72) Inventors: Pierre de Boucherville, Montreal (CA); Goran Matic, Beaconsfield (CA); Pierre Lepage, Drummondville (CA)

(73) Assignee: VIGILENT tELESYSTEMES iNC., Dorval (CA)

(21) Appl. No.: 13/980,445

(22) PCT Filed: Jan. 30, 2012
(86) PCT No.: PCT/CA2012/000088

§ 371 Date: Aug. 5, 2013

Related U.S. Application Data
(60) Provisional application No. 61/437,206, filed on Jan. 28, 2011.

(51) Int. Cl. A61B 19/00

(52) U.S. Cl. CPC A61B 19/5212
USPC 348/77

(57) ABSTRACT

The present document describes a system for installation in an operation room for providing a remote viewer/doctor with continuous visual access to the proceedings of a surgery (as a fixed target). The system comprises a camera comprising an internal processor for the processing of images/video, a moving platform on which the camera is provided, and pan and tilt motors for moving the camera head. A remote server sends pan/tilt commands for the pan and tilt motors and position commands for the moving platform to change positions. An external processor is provided at the system, which is dedicated to the processing of the pan and tilt commands without delay for panning and/or tilting the camera as the moving platform is changing positions at a rotating speed that allows the remote viewer to have un-interrupted visual access to the proceedings of the surgery.

### CROSS-REFERENCE TO RELATED APPLICATIONS

This application claims priority from U.S. provisional Application No. 61/437,206 filed on Jan. 28, 2011.

### BACKGROUND

(a) Field

The subject matter disclosed generally relates to a monitoring system used in surgery rooms. More particularly, the subject matter relates to a monitoring system for remotely monitoring the proceedings of a surgery in a surgery room.

(b) Related Prior Art

Cameras used for the remote monitoring of objects are generally adapted to pan (rotate about a vertical axis) and tilt (rotate about a horizontal axis) to follow a certain target. These cameras are used in many fields including security, satellites, airplanes, medical systems etc.

To facilitate the panning and tilting of the camera head, two independently actuated motor mechanisms are usually employed. The first motor is known as the “pan motor,” which facilitates the panning of the camera head. The second motor is known as a “tilt motor,” which facilitates the tilting of the camera head.

Examples of these cameras are shown in FIGS. 1a and 1b. These cameras are usually controlled remotely to pan and/or tilt the camera head to follow a certain target.

In existing systems, mechanical/optical zoom, focus, image processing (electronic zoom, image filtering etc.), and control of the pan and tilt commands are done by the same processor. Usually, this processor is provided in the camera, and gives the least priority to the processing of pan/tilt commands. Accordingly, the pan and tilt commands wait until a slot becomes available to be processed and sent for execution, which introduces delays in the panning and/or tilting of the camera head. The duration of these delays vary depending upon the busyness of the processor.

While undesirable, these delays do not create life threatening situations in the fields of security and airplanes, where the lost target may be found and followed again. However, loss of the view in a camera used in medical systems e.g. telepresence etc. is unacceptable, especially when the system is used to assist/perform a surgery remotely.

Accordingly, there is a need for a system which allows for adjusting the position of a camera remotely without delay, and without losing the target during the adjusting.

### SUMMARY

According to an embodiment, there is provided monitoring system in communication with a remote server for providing a remote viewer with visual access to a fixed target in a surgery room. The monitoring system comprises: a moving platform; a pan motor and a tilt motor; a camera rotatably attached to the moving platform using the pan motor and the tilt motor, the camera comprising an internal processor for processing images, the camera providing the visual access to the fixed target; an external processor dedicated for processing pan and tilt commands as soon as the commands are received from the remote server for controlling rotation of the pan motor and the tilt motor; wherein the pan and/or tilt commands are executed simultaneously as the moving platform is changing positions at a rotation speed that provides the visual access to the fixed target substantially un-interruptedly as the moving platform is moving.

According to another embodiment, the moving platform comprises: a first motor and a second motor; a first arm having a first end and a second end opposite the first end, the first arm rotatably attachable to a ceiling of the surgery room using the first motor provided at the first end of the first arm; a second arm having a first and a second end opposite the first end, the second arm rotatably attached at the first end thereof to the second end of the first arm, using the second motor; wherein the camera is attached to a second end of the second arm.

According to a further embodiment, the external processor is provided inside the second arm.

According to another embodiment, communication between the remote server on one side and the camera and the external processor on the other side are sent over a single communication link.

According to a further embodiment, communication between the remote server and the camera is effected using a first protocol, and communication between the remote server and the external processor is effected using a second protocol different from the first protocol.

According to another embodiment, the monitoring system further comprises an Ethernet switch for directing data between the remote server, the external processor and the camera based on which one of the first and second protocol of communication the data is encoded.

According to a further embodiment, the monitoring system further comprises a first limit switch dedicated to the pan motor, and a second limit switch dedicated to the tilt motor, the first limit switch and the second limit switch are for re-positioning the pan motor and the tilt motor to a reference point.

According to another embodiment, the pan motor and the tilt motor are step motors.

According to a further embodiment, the external processor keeps track of the position of each motor for processing of future commands.

According to another embodiment, the monitoring system further comprises an H-bridge for controlling the voltage applied on the pan motor and the tilt motor for rotating the pan motor and the tilt motor at desired speeds and directions.

According to an embodiment, there is provided a monitoring system in communication with a remote server for providing a remote physician with visual access to a patient in a surgery room. The monitoring system comprising: a moving platform defining a first arm for attachment to a ceiling and a second arm rotatably attached to the first arm; a pan motor and a tilt motor; a camera rotatably attached to the second arm using the pan motor and the tilt motor, the camera comprising an internal processor for processing images, the camera providing the visual access to the patient; an external processor provided in one of the first arm and second arm, the external processor being dedicated for processing pan and tilt commands received from the remote server independently of the processing of the images by the internal processor; wherein the pan and/or tilt commands are executed simultaneously as the platform is changing positions at a rotation speed that provides the visual access to the patient from different positions un-interruptedly and as the moving platform is moving.

According to an embodiment, there is provided a method for providing a remote viewer with visual access to a fixed target in a surgery room using a camera mounted on a moving platform using a pan motor and a tilt motor, the method comprising: processing images captured by the camera in an internal processor of the camera; receiving position commands for changing a position of the moving platform; receiving pan and/or tilt commands for changing an orientation of the camera; processing the pan and/or tilt commands in a an external processor independently of the processing of images; executing the pan and/or tilt commands simultaneously as the position commands are executed, at a rotation speed that provides the visual access to the fixed target un-interruptedly as the moving platform is moving.

According to another embodiment, the method further comprises providing the external processor in the moving platform.

According to a further embodiment, the method further comprises communicating with a remoter server by the external processor and the internal processor over a single communication link.

According to another embodiment, the method further comprises encoding data associated with the external processor using a first communication protocol; and encoding data associated with the internal processor using a second communication protocol different from the first communication protocol.

According to a further embodiment, the method further comprises: switching data received via the single communication link to one of the internal processor and external processor based on which one of the first and second protocol of communication the data is encoded.

According to another embodiment, the method further comprises: keeping track of a current position of the pan motor and the tilt motor; and implementing a shortest path algorithm in the external processor for executing future pan motor and tilt commands using the shortest path to a destination.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIGS. 1a and 1b illustrates examples of conventional cameras which are equipped with pan/tilt functionalities;

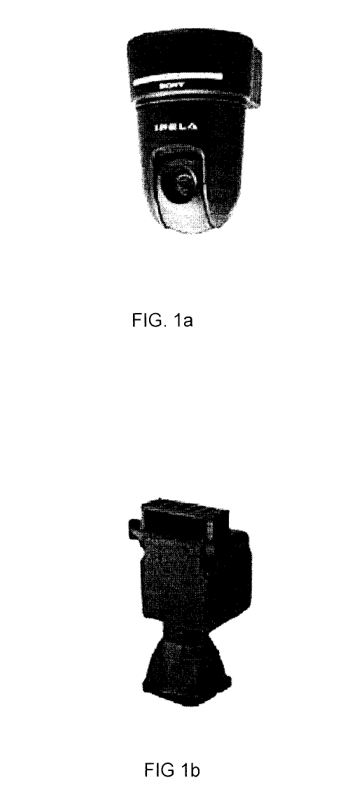

FIG. 2 illustrates an exemplary implementation of a moving platform which may be attached to a ceiling to monitor an object located underneath, in accordance with an embodiment;

FIG. 3 illustrates an example of a camera provided with pan and tilt motors;

FIG. 4 is a side view of a surgery room equipped with the moving platform of FIG. 2;

FIG. 5 is a bottom view of the moving platform of FIG. 2 showing the platform in different positions while monitoring the same target;

FIG. 6 is an exemplary block diagram of an electronic system 20 for processing the pan and tilt commands received from the remote device, in accordance with an embodiment; and

FIG. 7 is a flowchart of a method for providing a remote viewer with visual access to a fixed target in a surgery room.

It will be noted that throughout the appended drawings, like features are identified by like reference numerals.

### DETAILED DESCRIPTION

The present document describes a system for installing in an operation room for providing a remote viewer/doctor with continuous visual access to the proceedings of a surgery (as a fixed target). The system comprises a camera comprising an internal processor for the processing of images/video, a moving platform on which the camera is provided, and pan and tilt motors for moving the camera head. A remote server sends pan/tilt commands for the pan and tilt motors and position commands for the moving platform to change positions. An external processor is provided at the system, which is dedicated to the processing of the pan and tilt commands without delay for panning and/or tilting the camera as the moving platform is changing positions at a rotating speed that allows the remote viewer to have un-interrupted visual access to the proceedings of the surgery.

Cameras used in medical systems for telepresence or the like are often set to monitor a fixed target to allow a doctor to supervise and/or assist in performing the surgery remotely. To do so, the doctor supervising the surgery has to have un-interrupted (continuous) visual access to the proceedings of the surgery. However, the path is not always clear for the camera due to the movements of the nurses/doctors in the operation room. Therefore, when the view is blocked, the position of the camera needs to be changed in order to have a clear perspective of the patient.

An example of the existing systems used in controlling the position of the camera is described in U.S. Pat. No. 6,434,329 which is incorporated herein by reference in its entirety. In such system, processing of the images and the pan/tilt commands is performed by the processor of the camera. As discussed above, priority is given to the image processing and the pan/tilt commands are often delayed which introduces undesired visual interruptions to the remote viewer (doctor).

The following embodiments describe a system which allows for controlling the pan/tilt of a camera provided on a moving platform to continuously monitor a fixed object/target from different positions, and as the platform is moving without latency and without losing the line of sight. In an embodiment, the system reduces the delays in the processing of pan/tilt commands by processing the pan/tilt commands at least partially in parallel to and independently from the image processing. The pan and tilt motors execute the pan and tilt commands at a speed that compensates for the movements of the platform in real time so as to keep monitoring the same target even as the platform is changing positions.

In order to achieve this goal, there is a need for a camera system which can perform both 1) a fast processing of the pan and tilt commands to send these commands to the pan/tilt motors, and 2) a fast response by the pan/tilt motors in order to compensate for the movements of the platform.

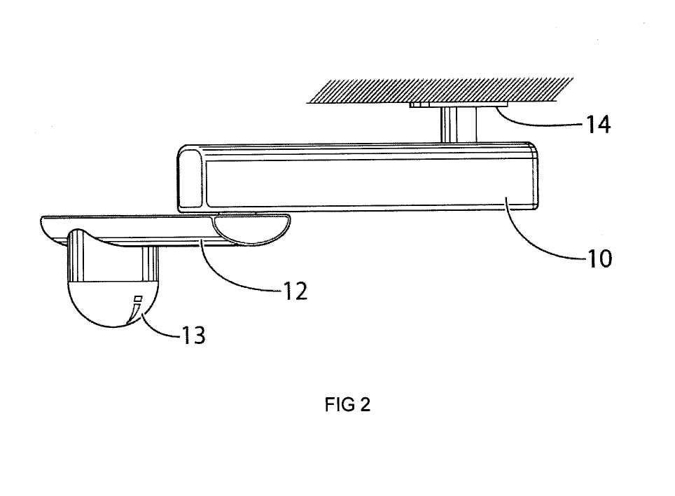

FIG. 2 illustrates an exemplary implementation of a moving platform which may be attached to a ceiling to monitor an object located underneath, in accordance with an embodiment. It is to be noted that the embodiments are not limited to this implementation but may be implemented with a variety of moving platforms without departing from the scope of the description.

In the example shown in FIG. 2, a platform is provided which includes a first (or main) arm 10 and a second (or secondary) arm 12. The arms are rotatably attached to each other, as illustrated. In an embodiment, the arms used are of the type S.C.A.R.A. The main arm 10 may be attached to the ceiling (or a wall) using a ring 14 or any suitable attachment. The main axis of rotation is that around which the main arm 10 rotates around the ring 14. The secondary axis of rotation is that around which the second arm 12 rotates around the first arm 10. The camera 13 may be attached at the free end of the second arm 12, as illustrated in FIG. 2.

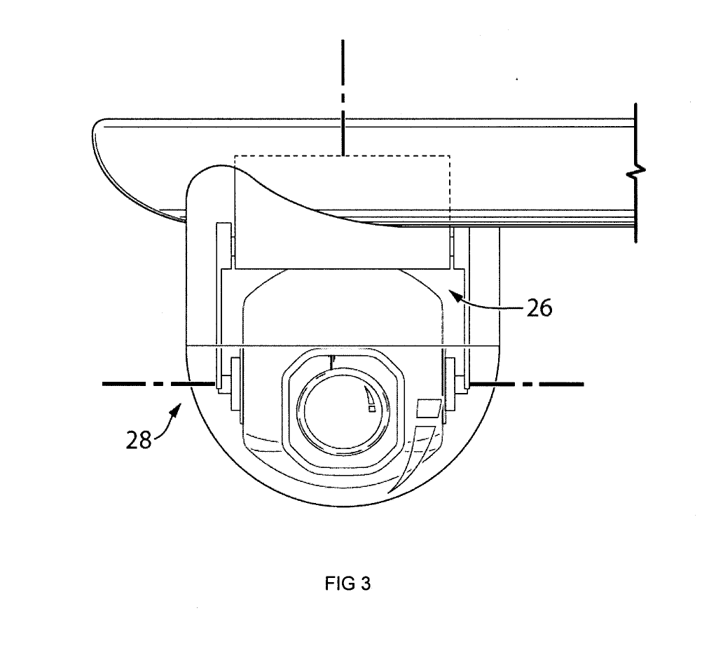

FIG. 3 illustrates an example of a camera 13 that may be used in the present embodiments. The camera is provided with a pan motor 26 and a tilt motor 28. The pan and tilt motors 26 and 28 are chosen to have sufficient rotation speed to compensate for the rotation of the arms, in order to continuously monitor the same target (fixed target) as the arms are moving.

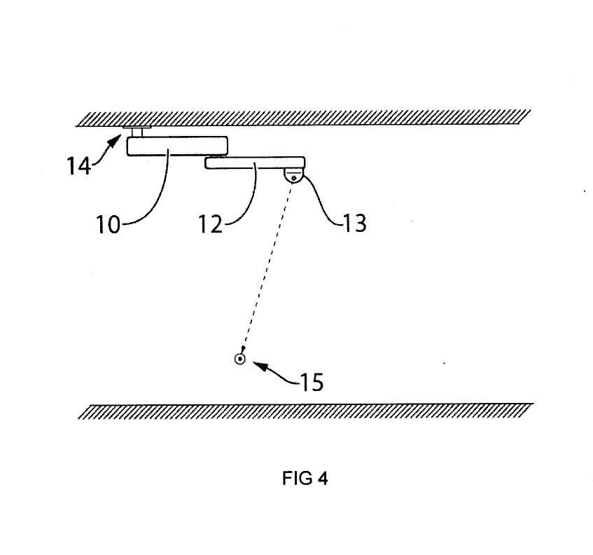

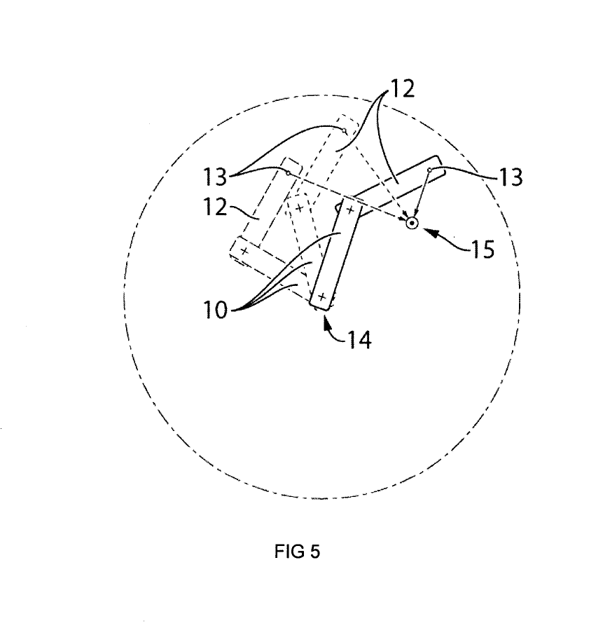

FIG. 4 is a side view of a surgery room equipped with the moving platform of FIG. 2. As shown in FIG. 4, the moving platform is attached to the ceiling of a surgery room to monitor a fixed target 15, which is usually an area of the body of a patient where the operation is being performed. FIG. 5 is a bottom view of the moving platform of FIG. 2 showing the platform in different positions while the camera is capturing the same target 15.

In an embodiment, control of the pan and tilt motors is effected remotely, whereby a remote device (computer) computes the direction of rotation and speed of each of the pan and tilt motors of the camera with respect to the movements of the platform so that the camera keeps monitoring the same target as the platform is moving. The remote device sends the rotation direction and rotation speed of each motor as pan and tilt commands to an electronic system in the surgery room for processing the commands and executing them in the motors.

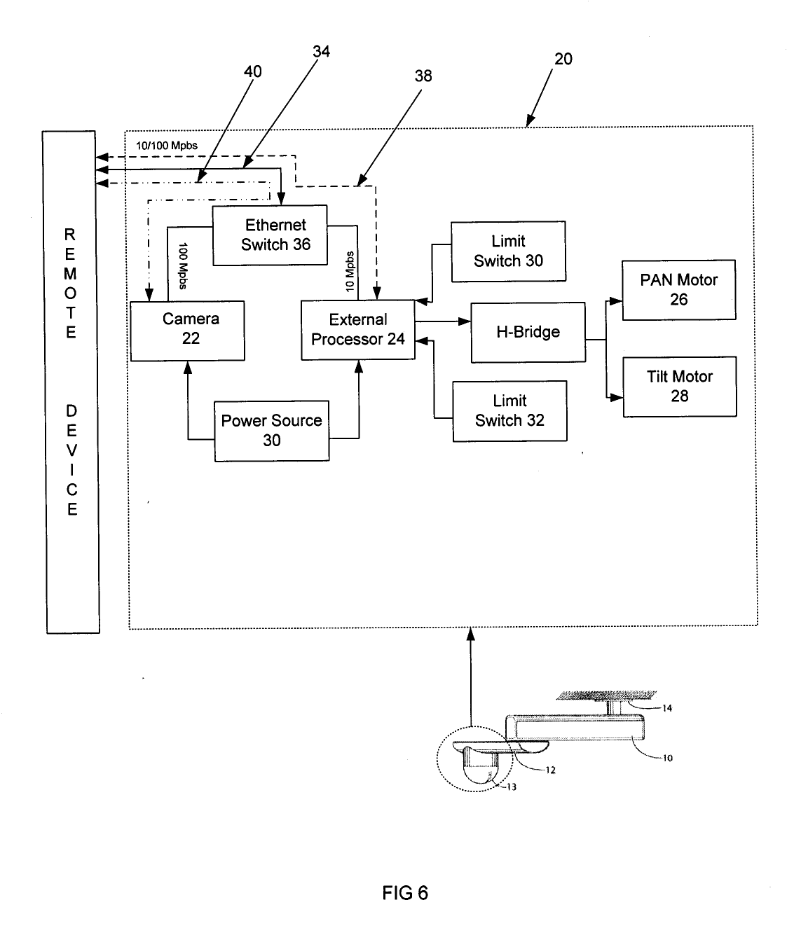

FIG. 6 is an exemplary block diagram of an electronic system 20 for processing the pan and tilt commands received from the remote device. In an embodiment, the system 20 is provided in the second arm 12 (or also in the main arm 10) of the moving platform, as indicated in FIG. 6. The system 20 includes a camera 22. In an embodiment, the camera 22 is a Sanyo VCC-HD4600 camera including an internal processor dedicated for the processing of video/images. In the present embodiment, the system 20 comprises an external processor 24 dedicated to processing the pan and tilt commands for controlling the pan motor 26 and the tilt motor 28 of the camera 22. In cases where the power voltage of the external processor 24 is different from that of the camera 22, a local power source 30 may be provided to generate different voltages to the different devices.

Communication between the system 20 and the remote device may be effected over a communication network such as WAN, LAN, the internet, a wireless network, an optical network or any combination of the above.

In an embodiment, the system 20 comprises two limit switches 30 and 32 in communication with the external processor 24 for re-positioning the pan motor 26 and tilt motor 28 to a reference position when the system is turned off/on so that the motors start from the same position every time the system is powered on. In an embodiment, the switches may use an IR (Infra-Red) source for re-positioning the pan and tilt motors to the reference position.

Due to the fact that, in the existing systems, processing of the pan/tilt commands and the image processing are performed by the internal processor of the camera, only one communication link 34 exists between the camera in the surgery room and the remote device used for monitoring the proceedings of the surgery. Embodiments of the invention utilize the same communication link 34 to communicate with both the camera 22 and the external processor 24. In the present embodiment, a TCP/IP protocol is used to manage the communication between the remote device and each of the camera 22 and the external processor 24 using an Ethernet switch 36.

The Ethernet switch acts like a hub to redirect the messages received from the remote device to the external processor 24 and the internal processor camera 22 and vice versa. Every time a processor sends a message (command) on the communication link 34, it adds its own IP address as well as the destination IP address. This way, all the other processors connected on the communication link 34 can know if the message is for them and from whom it comes. In an embodiment, the external processor 24 comprises an IP stack and communicates with the remote device using a protocol 38 which is different from the protocol 40 used by the camera 22. Accordingly, the communication link 34 transmits information encoded in accordance with a first protocol 38 for communication with the external processor 24 and a second protocol 40 for communication with the camera 22.

In addition to the pan and tilt commands, the remote device sends to the external processor the Settings of TCP/IP of the board (DHCP vs. Static, IP address, Subnet and Gateway), desired velocity profile for Pan and Tilt motors, and desired current profile for Pan and Tilt motors. In addition to this information, the external processor may send to the remote device the Ethernet MAC address, Homing status (in progress, done, error), and ID and Version of the product.

The external processor 24 processes the PAN/TILT commands at least partially in parallel to the image processing performed by the internal processor of the camera 22. Whereby, the delay is substantially eliminated, and the camera 22 can keep on monitoring the same target without interruption even when the arms are moving. In an embodiment, an H-bridge is connected between the external processor 24 and the pan and tilt motors (26 and 28). The H-bridge enables a voltage to be applied across a load in either direction to allow the pan and tilt motors to run forwards and backwards.

In an embodiment, rotation of the pan motor 26 is endless, while rotation of the tilt motor 28 is limited to a rotation of 180°. In an embodiment, a shortest path algorithm is used in order to create shortcuts and rotate the camera to the desired position in less time. For example, if it is determined that the pan motor needs to make a rotation of 270° to the right, the algorithm, depending on the situation, may substitute the 270° rotation to the right by a rotation of 90° to the left to reach the desired destination.

In an embodiment, the pan motor and the tilt motor are step motors which may be controlled by a series of polarization sequences. Each sequence makes the motor move one step forward or backward. In an embodiment, the external processor keeps track of the position of each motor in order to determine whether the motor should move forward or backward to execute the commands received from the remote device. In an embodiment, the tilt motor 28 performs 960 ticks to rotate 180° and has a speed that varies between 38 and 199 degree/second. By contrast, the pan motor performs 1920 ticks to rotate 360°, and has a speed that varies between 38 and 398 degree/second. In an embodiment, the speed and current limit of the motors can be accelerated and decelerated between the start and end points.

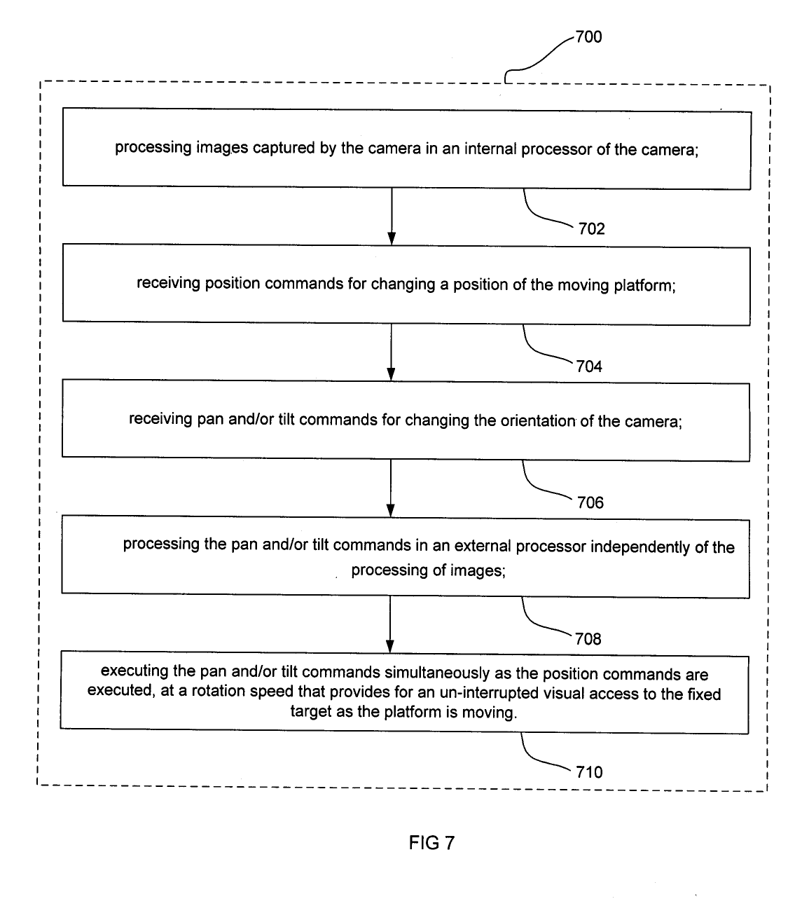

FIG. 7 illustrates a flowchart of a method 700 for providing a remote viewer with visual access to a fixed target in a surgery room. The method 700 is for providing a remote viewer with visual access to a fixed target in a surgery room using a camera mounted on a moving platform using a pan motor and a tilt motor. The method 700 comprises the step 702 of processing images captured by the camera in an internal processor of the camera. Moreover, the method 700 comprises the step 704 of receiving position commands for changing a position of the moving platform. Additionally, the method 700 comprises the step 706 of receiving pan and/or tilt commands for changing the orientation of the camera. The method 700 further comprises the step 708 of processing the pan and/or tilt commands in an external processor independently of the processing of images. Also, the method 700 comprises the step 710 of executing the pan and/or tilt commands simultaneously as the position commands are executed, at a rotation speed that provides for an un-interrupted visual access to the fixed target as the platform is moving.

According to an embodiment, the method 700 may further comprise a step of providing the external processor in the moving platform.

According to another embodiment, the method 700 may further comprise the step of connecting the external processor and the internal processor to a single communication link for communicating with the remote viewer.

According to a further embodiment, the method 700 may further comprises the steps of encoding data associated with the external processor using a first communication protocol; and of encoding data associated with the internal processor using a second communication protocol different from the first protocol.

According to another embodiment, the method 700 may further comprise the step of switching data received via the single communication link to one of the internal processor and external processor based on the protocol of communication with which the data is encoded.

According to a further embodiment, the method 700 may further comprise the steps of keeping track of the current position of the pan and tilt motors; and of implementing a shortest path algorithm in the external processor for executing future pan and tilt commands using the shortest path to a destination.

Embodiments can be implemented as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server over the network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

While embodiments have been described above and illustrated in the accompanying drawings, it will be evident to those skilled in the art that modifications may be made without departing from this disclosure. Such modifications are considered as possible variants comprised in the scope of the disclosure.

1. A monitoring system in communication with a remote server for providing a remote viewer with visual access to a fixed target in a surgery room, the monitoring system comprising:
a moving platform;
a pan motor and a tilt motor;
a camera rotatably attached to the moving platform using the pan motor and the tilt motor, the camera comprising an internal processor for processing images, the camera providing the visual access to the fixed target;
an external processor dedicated for processing pan and tilt commands as soon as the commands are received from the remote server for controlling rotation of the pan motor and the tilt motor;
wherein the pan and/or tilt commands are executed simultaneously as the moving platform is changing positions at a rotation speed that provides the visual access to the fixed target substantially un-interruptedly as the moving platform is moving.

2. The monitoring system of claim 1, wherein the moving platform comprises:
a first motor and a second motor;
a first arm having a first end and a second end opposite the first end, the first arm rotatably attachable to a ceiling of the surgery room using the first motor provided at the first end of the first arm;
a second arm having a first and a second end opposite the first end, the second arm rotatably attached at the first end thereof to the second end of the first arm, using the second motor;
wherein the camera is attached to a second end of the second arm.

3. The monitoring system of claim 2, wherein the external processor is provided inside the second arm.

4. The monitoring system of claim 1, wherein communication between the remote server on one side and the camera and the external processor on the other side are sent over a single communication link.

5. The monitoring system of claim 4, wherein communication between the remote server and the camera is effected using a first protocol, and communication between the remote server and the external processor is effected using a second protocol different from the first protocol.

6. The monitoring system of claim 5, further comprising an Ethernet switch for directing data between the remote server, the external processor and the camera based on which one of the first and the second protocol of communication of the data is encoded.

7. The monitoring system of claim 1, further comprising a first limit switch dedicated to the pan motor, and a second limit switch dedicated to the tilt motor, the first limit switch and the second limit switch are for re-positioning the pan motor and the tilt motor to a reference point.

8. The monitoring system of claim 1, wherein the pan motor and the tilt motor are step motors.

9. The monitoring system of claim 8, wherein the external processor keeps track of the position of each motor for processing of future commands.

10. The monitoring system of claim 8, further comprising an H-bridge for controlling voltage applied on the pan motor and the tilt motor for rotating the pan motor and the tilt motor at desired speeds and directions.

11. A monitoring system in communication with a remote server for providing a remote physician with visual access to a patient in a surgery room, the monitoring system comprising:
a moving platform defining a first arm for attachment to a ceiling and a second arm rotatably attached to the first arm;
a pan motor and a tilt motor;
a camera rotatably attached to the second arm using the pan motor and the tilt motor, the camera comprising an internal processor for processing images, the camera providing the visual access to the patient;
an external processor provided in one of the first arm and second arm, the external processor being dedicated for processing pan and tilt commands received from the remote server independently of the processing of the images by the internal processor;
wherein the pan and/or tilt commands are executed simultaneously as the moving platform is changing positions at a rotation speed that provides the visual access to the patient from different positions un-interruptedly and as the moving platform is moving.

12. A method for providing a remote viewer with visual access to a fixed target in a surgery room using a camera mounted on a moving platform using a pan motor and a tilt motor, the method comprising:
processing images captured by the camera in an internal processor of the camera;
receiving position commands for changing a position of the moving platform;
receiving pan and/or tilt commands for changing an orientation of the camera;
processing the pan and/or tilt commands in an external processor independently of the processing of images;
executing the pan and/or tilt commands simultaneously as the position commands are executed, at a rotation speed that provides the visual access to the fixed target un-interruptedly as the moving platform is moving.

13. The method of claim 12, further comprising providing the external processor in the moving platform.

14. The method of claim 12, further comprising communicating with a remote server by the external processor and the internal processor over a single communication link.

15. The method of claim 14, further comprising:
encoding data associated with the external processor using a first communication protocol; and
encoding data associated with the internal processor using a second communication protocol different from the first communication protocol.

16. The method of claim 15, further comprising:
switching data received via the single communication link to one of the internal processor and external processor based on which one of the first and second protocol of communication the data is encoded.

17. The method of claim 15, further comprising:
keeping track of a current position of the pan motor and the tilt motor; and
implementing a shortest path algorithm in the external processor for executing future pan and tilt commands using the shortest path to a destination.
